(19) United States
(12) Patent Application Publication
(10) Pub. No.: US 2010/0047336 A1
(43) Pub. Date: Feb. 25, 2010

(54) INHIBITION OF BRAIN ENZYMES INVOLVED IN CEREBRAL AMYLOID ANGIOPATHY AND MACULAR DEGENERATION

(75) Inventors: Wolff Kirsch, Redlands, CA (US); Lance A. Liotta, Bethesda, MD (US); William Van Nostrand, East Setauket, NY (US); Harry V. Vinters, Venice, CA (US); Virginia A. Espina, Rockville, MD (US)

Correspondence Address: KNOBBE MARTENS OLSON & BEAR LLP, 2040 MAIN STREET, FOURTEENTH FLOOR, IRVINE, CA, 92614, US

(73) Assignees: LOMA LINDA UNIVERSITY MEDICAL CENTER, Loma Linda, CA (US); GEORGE MASON UNIVERSITY, Arlington, VA (US); STONY BROOK UNIVERSITY, Stony Brook, CA (US); THE REGENTS OF THE UNIVERSITY OF CALIFORNIA, Los Angeles, CA (US)

(21) Appl. No.: 12/542,635

(22) Filed: Aug. 17, 2009

Related U.S. Application Data
(63) Continuation of application No. 11/948,856, filed on Nov. 30, 2007, now abandoned.
(60) Provisional application No. 60/889,521, filed on Feb. 12, 2007.
(60) Provisional application No. 60/872,275, filed on Dec. 1, 2006.

(51) Int. Cl. A61K 9/127; A61K 31/7052; A61K 31/555; A61P 25/28

(52) U.S. Cl. 424/450; 514/44 A; 514/185

(57) ABSTRACT

A method of treating or inhibiting progress of dementia and/or macular degeneration in a mammal involves administering compositions containing siRNA to heme oxygenase-1 (HO-1) or heme oxygenase-2 (HO-2), a matrix metalloproteinase (MMP) inhibitor, a caspase inhibitor, or a metalloporphyrin in a manner that permits access to brain sites and/or the macula of the patient.

### RELATED APPLICATIONS

The present application claims the benefit of U.S. Provisional Application Ser. No. 60/889,521, filed Feb. 12, 2007, and U.S. Provisional Application Ser. No. 60/872,275, filed Dec. 6, 2006, the entirety of each of which is hereby incorporated by reference.

### STATEMENT REGARDING FEDERALLY SPONSORED R&D

The present invention was made with United States government support from the National Institute on Aging of the National Institutes of Health under Grant No. AG20948.

### REFERENCE TO SEQUENCE LISTING, TABLE, OR COMPUTER PROGRAM LISTING

The present application is being filed along with a Sequence Listing in electronic format. The Sequence Listing is provided as a file entitled SEQLIST_LOMAU_{—}170.TXT, created Nov. 29, 2007, which is 4 Kb in size. The information in the electronic format of the Sequence Listing is incorporated herein by reference in its entirety.

### BACKGROUND OF THE INVENTION

The loss of cognitive ability in the elderly is a very frequent problem for which no effective therapy has been yet devised. The commercial potential of an invention that addresses the loss of cognitive ability in the elderly is enormous. Delaying cognitive loss in the elderly by even a few years would save billions of dollars as well as preserving dignity of the aged. Current therapy of the vascular aspect of Alzheimer's disease-cerebral amyloid angiopathy (CAA)—has not been well-developed, and is ineffective.

In a significant number of dementia cases, the cause for loss of cognitive ability in the elderly is the reaction of brain to small microbleeds from tiny capillaries and arterioles. The brain has a violent response to blood outside of the blood vessels and this response far exceeds the size of the hemorrhage. The cause of this violent response is HO-1. HO-1 is activated by the presence of blood, which causes degradation of HO-1 to iron, carbon monoxide and bilirubin. These products are toxic to neurons and glia.

Heme oxidase has been inhibited in experimental brain hematomas by tin-mesoporphyrin with beneficial effects to the brain. (Koeppen et al., J. Neuropathol and Exp. Neurol, 63(6):587-597 (June 2004); and Wagner et al., Cell Mol. Biol. (Noisy-le-grand), 46(3):597-608 (May 2000), both of which are hereby incorporated by reference.) Other attempts have been made to inhibit HO-1 and HO-2 with protease inhibitors and there is one report of using a small interfering RNA (siRNA) to inhibit lung heme oxygenase activity by nasal administration. (Appleton et al., Drug Metab. Dispos., 27(10):1214-1219 (October 1999), hereby incorporated by reference.)

The amyloid-beta peptide (Aβ) has been shown to induce the synthesis, release and activation of MMP-9 in murine cerebral endothelial cells, resulting in increased extracellular matrix degradation. Studies using a transgenic mouse model for CAA showed extensive MMP-9 immunoreactivity in CAA-vessels with evidence of microhemorrhage in the transgenic mice, but not in corresponding control animals. (Lee et al., Annals of Neurology 54(3):379-382 (September 2003).

Drusen are extracellular deposits that lie beneath the retinal pigment epithelium (RPE) and are the earliest signs of age-related macular degeneration (AMD). Recent proteome analysis demonstrated that amyloid β (Aβ3) deposition was specific to drusen from eyes with AMD. Yoshida et al., J. Clin. Invest., 115:2793-2800 (1995).

Using small interfering RNA (siRNA) to eliminate caspase-2 expression, Lassus and co-workers (Lassus et al., 2002. “Requirement for caspase-2 in stress-induced apoptosis before mitochondrial permeabilization.” Science 297(5585):1352-4) show that caspase-2 is essential for stress-induced apoptosis in several cell lines. They also demonstrate that caspase-2 is necessary for the permeabilization of mitochondria and the release of the apoptotic factors cytochrome c and Smac/Diablo. Caspase-2 was shown to be required for the translocation of Bax to mitochondria, previously the earliest detectable change in the apoptotic machinery. These findings are consistent with other studies showing that caspase-2 acts upstream of the release of apoptotic factors from mitochondria 3-5. In sum, these results suggest that caspase-2, and not caspase-9, is the most apical caspase in stress-induced apoptosis, and that caspase-2 represents a critical new target for inhibiting the intrinsic apoptotic pathway in neurons.

RNA interference (RNAi) is a potentially powerful research tool for a wide variety of gene-silencing applications (Aoki, 2003; Holen, 2003; McManus, 2002; Scherr, 2003). Possible repercussions of RNAi in mammals are its use in the fight against certain diseases, such as cancer or virus and parasite infections (Aoki, 2003), as well as in the analysis of problems in cell and developmental biology (Fjose, 2001): there are, for example, many efficient human and murine siRNA sequences against members of apoptotic pathways, such as caspase-1, -2, -3, -8, and Fas (Zender, 2004).

RNAi can also be used to study the functions and interactions of genes (Bosher, 2000). siRNAs are easily synthesized and used to silence genes in cell cultures, and it is possible that silencing cell lines will be obtained (Paul, 2002; Svoboda, 2000). One of the earliest uses of RNAi technology in drug development has been its application in functional genomic analyses. During these studies many components of complex pathways have been identified and isolated and their relevance to various drug discovery applications has been assessed (Shuey, 2002).

RNAi can be used as a tool to identify possible novel targets in drug discovery. This approach has several advantages: it permits rapid target identification and processing and does not depend on preexisting knowledge of target biology. Using bioinformatics, libraries of designed siRNAs (several different siRNAs oligos per gene) can be used to elucidate novel targets for any biological pathway. This method allows for the functional analysis of thousands of genes simultaneously, is highly reproducible, and requires small amounts of siRNA oligos. This procedure allows for high-throughput testing of potential targets without compromising high specificity and sensitivity (Xin, 2004). siRNAs could also represent the next generation of antiviral therapeutics, and DNAs encoding siRNAs should be useful in various forms of gene therapy (Zamore, 2003). The activation of siRNAs appears to be short-lived in mammals. They are sequence-specific natural cellular products, do not produce toxic metabolites, have a long life-span in cell culture and calf serum, and are efficient even in low concentrations (Zamore, 2003; Zender, 2004).

Despite active work by drug firms on anti-dementia drug programs, the amyloid target has proven unfruitful. To date, there are no examples of MMP-, HO-1- or HO-2-specific knockdown in vivo for the purpose of preventing Alzheimer's disease.

### SUMMARY OF THE INVENTION

Methods and compositions for the prevention and treatment of cognitive deterioration and disorders are disclosed in accordance with preferred embodiments of the present invention. In preferred embodiments, the method of the present invention relates to regulation of the enzymes heme oxygenase-1 and -2 (HO-1 and HO-2, respectively) and matrix metalloproteinases (MMPs) for the prevention and treatment of cognitive deterioration and disorders.

In preferred embodiments, the present invention concerns methods for treating or inhibiting progress of dementia, especially dementia associated with microvascular hemorrhage.

A method of treating or inhibiting progress of dementia is disclosed in accordance with an embodiment of the present invention. The method comprises administering an siRNA to heme oxygenase-1 (HO-1) or heme oxygenase-2 (HO-2) in a manner that permits access to brain sites of said mammal.

A method of treating or inhibiting progress of dementia is disclosed in accordance with another embodiment of the present invention. The method comprises comprising administering an siRNA to HO-1 or HO-2 to the brain of said mammal.

A method of treating or inhibiting progress of dementia is disclosed in accordance with an embodiment of the present invention. The method comprises administering a matrix metalloproteinase (MMP) inhibitor in a manner that permits access to brain sites of said mammal.

A method of treating or inhibiting progress of dementia is disclosed in accordance with another embodiment of the present invention. The method comprises administering metalloporphyrin to a blood vessel endothelial cell receptor of said mammal, thereby inhibiting HO-1 and HO-2 and preventing weakening and bleeding in the vessel wall.

A method of treating or inhibiting progress of age-related macular degeneration (AMD) is disclosed in accordance with an embodiment of the present invention. The method comprises administering an siRNA to heme oxygenase-1 (HO-1) or heme oxygenase-2 (HO-2) in a manner that permits access to the retina or macula of said mammal.

A method of treating or inhibiting progress of AMD is disclosed in accordance with another embodiment of the present invention. The method comprises comprising administering an siRNA to HO-1 or HO-2 to the eye of said mammal.

A method of treating or inhibiting progress of AMD is disclosed in accordance with an embodiment of the present invention. The method comprises administering a matrix metalloproteinase (MMP) inhibitor in a manner that permits access to the retina or macula of said mammal.

### BRIEF DESCRIPTION OF THE DRAWINGS

FIG. 1 shows a comparison of gradient-echo (GE)-T₂* and susceptibility weighted imaging (SWI) for Brain Microhemorrhage (MH) Detection. The subject, an 88-year-old demented woman, has clearly defined multiple MH visible by SWI in a pattern consistent for CAA. The MH appear as “black holes” due to phase disturbances. Significantly more MH are detected by SWI in contrast to the few lesions noted with the current conventional sequence for MH detection GE-T₂*.

FIG. 2 shows a reverse phase protein microarray (RPPM) of protein from vitreous samples immunostained with anti-Heme-Oxygenase-1 antibody.

FIG. 3 shows Wilcoxon non-parametric comparison of means for the neo-vascular group and the non-neovascular group (labeled as “not”) (p=0.260).

### DETAILED DESCRIPTION OF THE PREFERRED EMBODIMENT

The present invention provides methods and compositions for inhibiting HO-1, HO-2, and MMPs, thereby slowing cognitive deterioration and treating or preventing dementia. Methods and compositions for treating or preventing dementia are disclosed in accordance with preferred embodiments of the present invention. Various embodiments of methods described herein will be discussed in terms of Alzheimer's disease-associated dementia. However, many aspects of the present invention may find use in treatment or prevention of other types of dementia.

Cerebral amyloid angiopathy (CAA), also known as congophilic angiopathy or cerebrovascular amyloidosis, is a disease of small blood vessels in the brain in which deposits of amyloid protein in the vessel walls may lead to stroke, brain hemorrhage, or dementia. In Alzheimer's disease, CAA is more common than in the general population, and may occur in more than 80% of patients over age 60. CAA is characterized by small blood vessel bleeding. This bleeding is caused when the amyloid protein A Beta 40 is targeted to the small blood vessel wall, where it activates HO-1 and triggers oxidative stress. The oxidative stress opens the vessel wall and causes microhemorrhages (MH).

Our ongoing study of individuals who have demented while under observation and undergoing special MR and proteomic testing has revealed a significant percentage associated with increasing microvascular hemorrhage. These hemorrhages have the distribution characteristic of CAA. The inventors have found that blood extravasating to the brain is extraordinarily toxic when degraded by the HO-1 and HO-2 enzymes. Red cells become lysed by complement, the hemoglobin is oxidized to met-hemoglobin and the latter is broken down into heme and globin. Heme is extraordinarily toxic and distributes rapidly along the small blood vessels and brain to turn on the gene for HO-1 (HO-2 is constitutive in the neurons). The breakdown of heme by heme oxygenase 1 and 2 results in the formation of carbon monoxide (CO), ferrous ion Fe⁺⁺, and biliverdin. Biliverdin is then converted to bilirubin.

HO-1 or HO-2 can be inhibited, for example, with a signal that turns off the gene for HO-1 or HO-2 production. For example, delivery of an siRNA to HO-1 or HO-2 in a liposome carrier targeted to an endothelial receptor located on an endothelial cell of a blood vessel in the brain inhibits HO-1 or HO-2 activation, thereby preventing MH due to A Beta 40.

In one embodiment, the present invention provides a method for treating or inhibiting progress of dementia in a mammal, comprising administering an siRNA to HO-1 or HO-2 in a manner that permits access to brain sites of said mammal. In one embodiment, the mammal is an elderly individual having fragile microvessels. In another embodiment, the mammal has Alzheimer's disease. In another embodiment, the mammal is a mammal susceptible to Alzheimer's disease

In some embodiments, siRNA can be endogenously expressed using, for example, a variety of siRNA expression systems. One alternative to direct introduction of short dsRNAs into cells uses the endogenous expression of siRNAs by various RNA polymerase III promoter systems (mouse U6, human H1, tRNA promoters) that allow transcription of functional siRNAs or their precursors (Lee, 2002; Scherr, 2003; Thompson, 2002). This way the produced siRNAs could be expressed for longer periods than exogenously introduced siRNAs, particularly in cells where the expression unit will integrate with the host genome (Brummelkamp, 2002; Shuey, 2002).

Zheng et al. (Zheng, 2004) have developed a dual-promoter siRNA expression system (pDual) in which a synthetic DNA encoding agene-specific siRNA sequence is inserted between two different opposing polymerase III promoters, the mouse U6 and human H1 promoters. Upon transfection into mammalian cells, the sense and antisense strands of the duplex are transcribed by these two promoters from the same template, resulting in an siRNA duplex with a uridine overhang on each 3′ terminus, similar to the siRNA generated by Dicer. These siRNAs can be incorporated into the RNA-induced Silencing Complex (RISC) without any further modifications and specifically and efficiently suppress gene functions.

In addition to pDual, Zheng et al. have developed a single-step PCR protocol that allows the production of siRNA expression cassettes in a high-throughput manner and they have constructed an arrayed siRNA expression cassette library that targets about 8000 genes with two sequences per gene (Zheng, 2004). Injection of plasmid DNA expressing long cytoplasmic dsRNA induces efficient RNAi in nonembryonic mammalian cells without stress response pathways. This system allows simultaneous expression a large number of siRNAs from a single precursor dsRNA, and longer dsRNA could include more than one message in a single construct.

Recently, vectors have been investigated which contain a cytomegalovirus (CMV) promoter and express long (about 500 nucleotides) dsRNAs, but these dsRNAs are not transported into cytoplasm and do not induce the interferon response (Foubister, 2003; Stanislawska, 2005). These dsRNAs are cleaved into siRNAs in the nucleus and are then transported to the cytosol, where they silence the target mRNA. This system is based on the polymerase II promoter and, although the CMV promoter is active in most cell types, these findings are a first step toward the use of tissue-specific polymerase II promoters. The potential advantage of this method is that there are numerous tissue-specific polymerase II promoters available (Foubister, 2003; Stanislawska, 2005).

A wide variety of siRNAs, including siRNAs to HO-1 and HO-2, are commercially available. A preferred source of siRNAs suitable for the purposes of the present invention is Dharmacon. Human HO-1 siRNA can also be purchased from Santa Cruz Biotechnology (catalog numbers sc-35554 and sc-44306) and Qiagen (catalog numbers SI02780533, SI02780995, SI00033089, and SI03111990). Human HO-2 siRNA is available from Santa Cruz Biotechnology (catalog number sc-35556). Custom siRNAs are also available from Dharmacon.

In some embodiment, the siRNA can be chemically synthesized. Chemical synthesis of siRNA is the most commonly used method to generate RNAi (Shuey, 2002). Alternatively, T7-transcribed siRNAs as well as siRNAs isolated from D. melanogaster embryo protein extracts were can be used (Shuey, 2002).

In some embodiments, siRNA at a concentration of between about 5 μg/ml to about 20 μg/ml can be administered. In some embodiments, siRNA can be administered at a concentration of about 5, 6, 7, 8, 9, 10, 11, 12, 13, 14, 15, 16, 17, 18, 19 or 20 μg/ml. It has been reported that high concentrations of dsRNAs (15 μg/ml) can induce inhibition of target gene expression in proliferating and differentiating cells in a nematode neuronal culture (Krichevsky, 2002). The siRNA can be administered by a variety of methods known in the art, including via physical delivery, such as, for example, electroporation, injection; chemical delivery, such as lipid- or liposome-mediated gene delivery, as discussed more fully below; and a peptide-based gene delivery system, MPG transfection (Plasterk, 2000; Simeoni, 2003).

Suitable delivery reagents for administration in conjunction with the present siRNA include, for example, a liposome such as, for example, a 1,2-dioleoyl-sn-glycero-3-phosphatidylcholine (DOPC) liposome; lipofectin; lipofectamine; cellfectin; or polycations (e.g., polylysine).

In one embodiment, the delivery reagent is a liposome or liposome carrier. In a preferred embodiment, the siRNA to HO-1 or HO-2 is in a DOPC liposome. In some embodiments, a liposome encapsulating the present siRNA comprises an immunoliposome. In other embodiments, a liposome encapsulating the present siRNA comprises a ligand molecule that can target the liposome to a particular cell or tissue at or near the site of angiogenesis. Ligands which bind to receptors prevalent in vascular endothelial cells, such as monoclonal antibodies that bind to endothelial cell surface antigens, are preferred.

In one embodiment, the liposome carrier is targeted to an endothelial cell receptor. Suitable endothelial cell receptors suitable for targeting in conjunction with the present siRNA include, for example, an LDL receptor, a VLDL receptor, and an LDL receptor-related protein (LRP). The endothelial cell receptor may be in the brain of a mammal. The endothelial receptor is preferably located on an endothelial cell of a blood vessel. In a preferred embodiment, the liposome is targeted to an LDL receptor. Preferably, the LDL receptor is located on an endothelial cell of a blood vessel in the brain.

The administration may be intravenous. Intravenous administration can provide access to brain sites because of the breakdown of the blood brain barrier secondary to the microhemorrhage. Intravenous administration can be accomplished, for example, with the use of an osmotic pump. In a preferred embodiment, HO-1/HO-2 siRNA-DOPC can be delivered to the target area using an ALZET® osmotic pump. The ALZET® osmotic pump requires no external connections or operator intervention during the entire delivery period. Thus, the use of osmotic pumps eliminates the need for frequent handling and repetitive injection schedules. ALZET® pumps have been shown to dependably deliver many types of drugs and are available in an assortment of sizes, flow rates and durations (some as long as four weeks of continuous infusion). ALZET® pumps are capable of delivering solutions with a viscosity of up to 100,000 cP (1 cP=1 mPas), which corresponds to roughly 200 times the viscosity of heavy weight engine oil. Thus, ALZET® pumps are suitable for delivery of liposomes. In addition, stereotactic intraventricular placement of cannulas can be used to administer siRNAs. Hoyer D. et al., J Receptors and Signal Transduction. 2006; 26:527-547.

Alternatively, siRNA can be introduced through the cerebrospinal fluid (CSF) to gain access to brain sites. When the administration of the siRNA is introduced through the CSF, the administration can be via, for example, lumbar puncture or ventricular puncture.

In one embodiment, the present invention provides a method for treating or inhibiting progress of dementia in a mammal, comprising administering an siRNA to HO-1 or HO-2 siRNA in a DOPC liposome intravenously using an ALZET® osmotic pump in a manner that permits access to brain sites of said mammal.

In another embodiment, the present invention provides a method for treating or inhibiting progress of dementia in a mammal, comprising administering an siRNA to, for example, HO-1 or HO-2 siRNA in a DOPC liposome intravenously using an ALZET® osmotic pump in a manner that permits access to brain sites of said mammal, wherein the liposome is targeted to an LDL receptor.

In another embodiment, the present invention provides a method for treating or inhibiting progress of dementia in a mammal, comprising administering an siRNA to, for example, HO-1 or HO-2 to the brain of said mammal. In one embodiment, the mammal is an elderly individual having fragile microvessels. In another embodiment, the mammal has Alzheimer's disease. In another embodiment, the mammal is a mammal susceptible to Alzheimer's disease.

In another embodiment, the present invention provides a method for treating or inhibiting progress of dementia in a mammal, comprising administering an siRNA to, for example, HO-1 or HO-2 siRNA to the brain of said mammal, wherein said siRNA is in a DOPC liposome delivered intravenously using an ALZET® osmotic pump.

In another embodiment, the present invention provides a method for treating or inhibiting progress of dementia in a mammal, comprising administering an siRNA to, for example, HO-1 or HO-2 siRNA to the brain of said mammal, wherein said siRNA is in a DOPC liposome delivered intravenously using an ALZET® osmotic pump, wherein the liposome is targeted to an LDL receptor.

A method of treating or inhibiting progress of dementia is disclosed in accordance with another embodiment of the present invention. The method comprises administering an MMP inhibitor in a manner that permits access to brain sites in the mammal. In one embodiment, the method comprises administering an MMP inhibitor that inhibits a particular MMP. In another embodiment, the method comprises administering a pan-MMP inhibitor. In a preferred embodiment, the method comprises administering an inhibitor to MMP-9.

Suitable MMP inhibitors useful in the present invention include, without limitation, broad-spectrum MMP inhibitors, pan-MMP inhibitors (i.e., an inhibitor of a wide range of MMPs), inhibitors that specifically recognize one or a combination of MMPs, including MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-24, MMP-25, MMP-26 and MMP-28. In a preferred embodiment, the MMP inhibitor is an inhibitor of MMP-9. MMP inhibitors are commercially available from, for example, Calbiochem or CHEMICON. In some embodiments, the MMP inhibitor is Batimastat, BAY 12-9566, BMS-275291, Marimastat, metastat, MM1270(B), or Prinomastat.

The MMP inhibitor may be an siRNA to an MMP. For example, the MMP inhibitor may be an siRNA to an MMP selected from the group consisting of MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-24, MMP-25, MMP-26 and MMP-28. In a preferred embodiment, the MMP inhibitor is an siRNA to MMP-9. In other embodiments, the MMP inhibitor is a combination of siRNAs to a combination of MMPs. As discussed above, siRNAs are commercially available and can also be custom ordered from Dharmacon. siRNAs to an MMP can be administered to a mammal using a liposome carrier as described above. In addition, an osmotic pump may be used to deliver the siRNA.

A method of treating or inhibiting progress of dementia is disclosed in accordance with another embodiment of the present invention. The method comprises administering a caspase inhibitor in a manner that permits access to brain sites in the mammal. In one embodiment, the method comprises administering a caspase inhibitor that inhibits a particular caspase. In another embodiment, the method comprises administering a pan-caspase inhibitor. In a preferred embodiment, the method comprises administering an inhibitor to casepase-2.

Caspase inhibitors may provide at least two levels of protection for neurons that are undergoing apoptosis through blocking and reversing the death program. Caspase inhibitors may also inhibit the cleavage of multiple intra and extra neuronal substrates, including amyloid components, degradation of which may generate toxic fragments.

A wide variety of caspase inhibitors are commercially available and useful in the present invention. They include, for example, IDN-1965, active-site mimetic peptide ketones such as zVAD-FMK, and IDN-6556. The broad-range caspase inhibitor IDN-1965 has been employed in continuous infusion studies for blocking cardiac damage during heart failure in a murine model. Treatment with IDN-1965 effectively reduced caspase 3-like activity and terminal dUTP nick end-labeling-positive myocytes, each by 90%. The treatment appeared to eliminate the 30% mortality seen in vehicle-treated mice. Caspases, cysteinyl aspartate-specific proteases, are important targets for therapeutics intended to inhibit apoptotic pathways. Broad spectrum caspase inhibitors, such as the active-site mimetic peptide ketones (i.e. zVAD-FMK), while not ideal compounds for clinical applications, have been highly effective in animal models in reducing cell death after ischemia in multiple tissues, demonstrating that caspase inhibitors have great promise for improving outcomes after organ transplantation, cardiac arrest and stroke. Also nonselective caspase inhibitors have decreased apoptosis in animal models of amyotrophic lateral sclerosis, Parkinson's disease, and sepis. Idun Pharmaceutical's IDN-6556, a broad spectrum caspase inhibitor, is showing promise in human trials for preserving liver function during hepatitis C virus infection without exhibiting serious side-effects, validating the use of caspase inhibitors in humans.

The caspase inhibitor may be an siRNA to a caspase. For example, the caspase inhibitor may be an siRNA to an caspase selected from the group consisting of caspase-1, caspase-2, caspase-3, caspase-4, caspase-5, caspase-6, caspase-7, caspase-8, caspase-9, caspase-10, caspase-11, caspase-12, and caspase-13. In a preferred embodiment, the caspase inhibitor is an siRNA to caspase-2. In other embodiments, the caspase inhibitor is a combination of siRNAs to a combination of caspases. As discussed above, siRNAs are commercially available and can also be custom ordered from Dharmacon. siRNAs to an caspase can be administered to a mammal using a liposome carrier as described above. In addition, an osmotic pump may be used to deliver the siRNA.

A method of treating or inhibiting progress of dementia is disclosed in accordance with another embodiment of the present invention. The method comprises administering metalloporphyrin (Mp) to a blood vessel endothelial cell receptor of said mammal, thereby inhibiting HO-1 and HO-2 and preventing weakening and bleeding in the vessel wall.

As discussed in the background of invention section, age-related macular degeneration shares the feature of Aβ deposition with Alzheimer's Disease. The applicants also note that excess vascularization is also associated with macular degeneration. It is believed that the excess vascularization itself is not the cause of the damage to the macula and resulting deterioration in vision. Rather, leakage from the excess blood vessels can occur creating microhemorrhages from these vessels. Such microhemorrhages are believed to cause damage to the macula in a manner analogous to the damage caused to cerebral tissue in CAA. Furthermore, elevated levels of HO-1 have been vitreous humor in patients suffering from “wet” macular degeneration (see, Examples below).

Macular degeneration can be treated in a manner that will reduce microhemorrhages and/or reduce the toxicity of the materials released in the microhemorrhages. Thus, a composition containing active ingredient for this purpose can be administered in any manner that permits access to the macular tissue. For example, the compositions can be injected directly into the vitreal tissue of the eye.

Active ingredients for treatment of macular degeneration can include any and all of the ingredients disclosed above in connection with treatment of CAA. Thus, the disclosure above in connection with treatment of CAA is applicable to treatment of macular degenerations. Compositions containing siRNA to heme oxygenase-1 (HO-1) or heme oxygenase-2 (HO-2), a matrix metalloproteinase (MMP) inhibitor, a caspase inhibitor, or a metalloporphyrin can all be used for this purpose. The concentrations and amounts of active ingredient will be in the same general range described above in connection with treatment of CAA; however, those having ordinary skill in the art can use well-known pharmacological techniques to optimize such concentrations and amounts. In addition, delivery vehicles and other inert ingredients can be incorporated into ophthalmic compositions for this purpose.

In some embodiments, laser capture microdissection (LCM) can be used to quantitate and profile gene expression as well as signal pathways at the cellular level. Highly sensitive protein arrays can be used to measure the activity state (for example, phosphorylation or cleavage) of more than one hundred proteins involved in signal pathways including stress, prosurvival and apoptosis. Phosphorylated forms of proteins such as, for example, Akt, readily measurable by this technology are very difficult to detect, much less quantitate, by immunohistochemistry. LCM provides the opportunity for the first time to quantitatively study the potential gradient of, for example, HO-1 protein emanating from the pathologic vessels or from specific cell types within the brain. Moreover, LCM can be employed to measure the levels of, for example, HO-1 and local effected pathways such as PI3 Kinase prosurvival pathways, Hypoxia mediated pathways, and apoptosis pathways.

A mouse model of wet macular degeneration (Jackson Labs) is available and can be used to test the effect of siRNA to, for example, HO-1, HO-2, MMP, a caspase inhibitor or a metalloporphyrin on retinal tissue, as described in the Examples below.

LCM can be used to carry out quantitative reverse phase protein microarray analysis of affected brain tissue normalized to total protein. For example, homozygous deletion sample cluster showed quantitative differential levels of Hemoxygenase-1, Matrix Metalloproteinase 9 (MMP-9), AMPKβ1 ser108, and PDGFRβ Y716. Microdissected samples were lysed and analyzed by Reverse Phase protein microarrays (RPA) to quantitate HO-1 as well as the activation state of cellular inflammatory signal pathways. The RPA array format has achieved detection levels approaching attogram amounts of a given analyte such as HO-1.

Third-generation PCR amplification chemistries can be used to detect amplifications for proof of HO-1 and HO-1 gene expression. An anti-HO-1 antibody can be used to detect HO-1 both histochemically and quantitatively. RPA technology applied to quantitative tissue microanalysis has the significant advantages for quantitative measurements of HO-1 gene expression.

### EXAMPLES

The following Examples are offered by way of illustration and not by way of limitation.

### Example 1

### Therapeutic Trial of HO-1, HO-2, MMP, Caspase Inhibitor and Metalloporphyrin Inhibitors in the CAA Mouse Model

A mouse model of CAA will be studied for the therapeutic effects of agents directed to brain HO-1, HO-2, MMP, caspase inhibitor or metalloporphyrin inhibition.

APP transgenic mice will be evaluated using neurologic, pathologic, and biochemical parameters. Both APPDutch (pure CAA) and APPswe (mixed parenchymal amyloid and CAA) transgenic mice will be evaluated. Dr. Jucker (Tübingen) will provide the transgenic and control mouse models. Mouse SWI-MR brain imaging will be conducted at 11.7T at LLUMC. The natural history and neurologic course of the transgenic mice will be defined as well as neuropathology and LCM gradient assays at LLUMC, George Mason University (GMU), and UCLA. Once the natural history and phenotype of the model has been established, treatment trials with candidate siRNAs (siRNA to HO-1, HO-2, MMPs, a caspase inhibitor, or metalloporphyrin) and Mps (tin-mesoporphyrin IX, for example) will be instituted.

There will be a total of 6 groups of study animals with an n=16 for each group, male=female. 96 mice will be studied over 2 years. There are a number of available transgenic mouse models that overexpress the Swedish mutation of APP (APPsw and APP23). These mouse models demonstrate features of human CAA, including spontaneous intracerebral hemorrhage (ICH), with increasing amounts of ICH after thrombolysis or anti-AP immunotherapy. CAA in an amyloid precursor protein transgenic mouse model (APP23 mice) leads to a loss of vascular smooth muscle cells, aneurysmal vasodilatation, and in rare cases, vessel obliteration and severe vasculitis. This weakening of the vessel wall is followed by rupture and bleedings that range from multiple, recurrent microhemorrhages to large hematomas. In the APP23 mice, the extracellular deposition of neuron-derived beta-amyloid in the vessel wall is the cause of vessel wall disruption, which eventually leads to parenchymal hemorrhage.

Mice will be operated on at 11 months of age, treated for 1 month with intraventricular siRNA, then tested for spatial memory status. Animals will be killed and after cold PBS perfusion, brains harvested, the cerebellums removed, and divided in the midline. One hemisphere will be placed in 70 percent ethanol, 10 percent PEG for immunohistochemical assays, the other snap frozen in liquid nitrogen for biochemical assays. The ethanol fixed hemisphere will be studied for immunohistochemistry for quantitation of the inflammatory response (reactive HO-1 immunopositive astrocytes, microglia included), amyloid deposition, and histochemical evident iron.

The tissue sections will be reviewed for the neuropathological features of treated transgenics, control transgenics and WT animals. Results of these immunohistochemical studies will form the basis for the number of brains to be studied by LCM.

The snap frozen hemispheres will be pulverized to create homogenized samples (˜15 mg), and 5 mg powder aliquots will be subjected to three different extraction procedures. The aliquots will be analyzed for the following. i) Carbon monoxide generation to determine global HO (HO-1, HO-2) activity, ii) quantitative RT-PCR to determine the number of transcripts of mRNA for HO-1 HO-2, Western blots for HO-1, HO-2 quantitative determination, iii) content of β-amyloid oligomers, total iron, and inflammatory cytokines. One of the aliquot of frozen brain powder (50 mg) will be used for determination of heme oxygenase activity measured by carbon monoxide generation. Outcomes ii) and iii) above will be measured. Results from the initial 48 animals will provide information regarding extent and effect of HO-1 gene knockdown to form the basis for dosimetry and siRNA composition, as well as the number of LCM studies to regionally profile the HO-1 gene in the second year of the study.

MR-SWI brain imaging of MCI and control participants at 3T correlated with sequential psychometric and serum proteomic examinations will be carried out in sufficient numbers to validate our hypothesis. SWI imaging and laser will capture microdissection of tissue gradients at a series of radial distances from amyloid microhemorrhages of proven CAA necropsied brains to interrogate the perifocal reactive zone for critical molecular interactions.

### Example 2

### Selection of Targeting siRNAs

This Example illustrates the selection of targeting siRNAs.

The sequences of targeting siRNAs, such as, for example, HO-1, HO-2, MMP, caspase inhibitor or metalloporphyrin targeting siRNAs, can be been checked for theoretical specificity against the mouse transcriptome by blast searches against the mouse genome using NCBI. For example, the following steps and guidelines can be taken to maximize success in siRNA target sequence selection. (1) Find the regions of a cDNA to choose target sequences. A target sequence is preferably specific to the target gene and shows little or no significant homology to any other genes. Using the blast search, regions of the target cDNA with no or low homology to other genes can be identified, from which candidate siRNA target sequences can be chosen. (2) A target sequence preferably starts with a “G” because RNA Polymerase III begins transcription with a “G” from the U6 promoter. (3) Preferably, avoid strings of four “Ts” in the designed hairpin. Four or five “Ts” is a stop signal for the transcription of Pol III and their presence in the designed hairpin will lead to premature transcriptional termination. (4) Avoid sequences containing KpnI or HindIII sites. KpnI and HindIII are used to digest the PCR products later on. Their presence in a target sequence will result in nonfunctional constructs. (5) Avoid sequences close to the ATG translational start codon. The region close to ATG on the mRNA may be associated with multiple proteins involved in translation that may interfere with RISC binding. A target sequence can also be selected from a 3″-UTR region. (6) Avoid sequences with internal repeats or palindromes. The presence of these structures will reduce the production of functional hairpins. (7) Use a sequence with a low G/C content, especially at its 3′ end. SiRNAs with lower G/C content are believed to yield better silencing. (8) Use a sequence with high specificity to the target gene. Target sequence candidates can be analyzed using the NCBI/Blast website to ensure that they do not significantly match any other gene sequence.

### Example 3

### Diagnosis of Cerebral Amyloid Angiopathy with SWI MR Imaging

This Example illustrates the use and advantages of SWI imaging for earlier and precise diagnosis of Cerebral Amyloid Angiopathy (CAA).

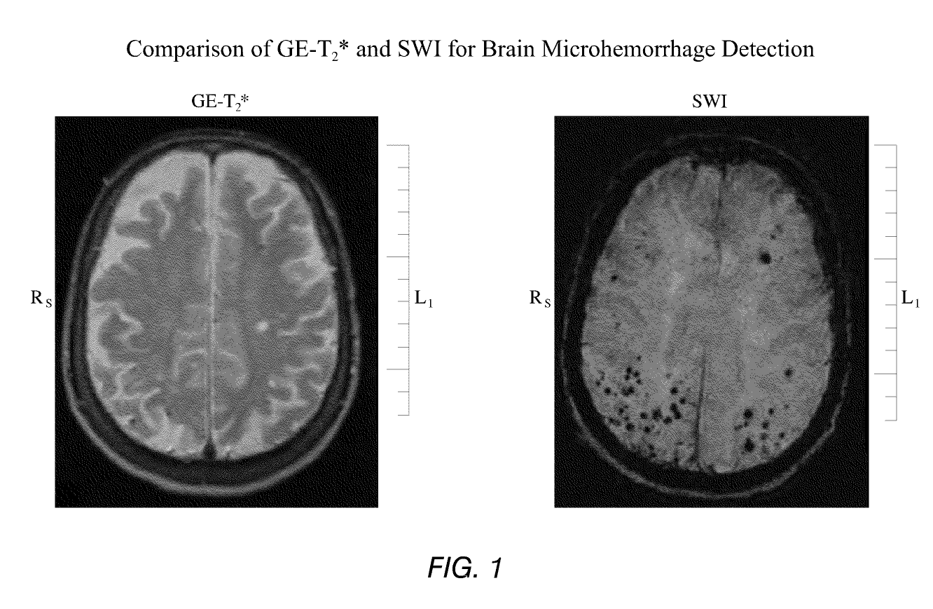

Mounting evidence indicates that CAA with secondary brain microhemorrhages (MH) plays an important yet underestimated role in the pathogenesis of sporadic late onset dementia. A small amount of extravasated blood in the brain results in an enlarging gradient of neuronal and neuropil damage termed the “perifocal reactive zone.” Rapid perivascular heme diffusion results in hyperexpression of brain heme oxygenase-1 (HO-1) with resulting free ferrous iron, carbon monoxide and biliverdin—all potentially neurotoxic at a volumetric distance from the MH. Studies in experimental animals have established that inhibition of hemorrhage-induced brain HO-1 by metalloporphyrins (Mps) provides neuronal protection. Thus, in view of the evidence for increasing microbleeds in the aging brain a therapeutic strategy directed towards inhibition of brain HO-1 warrants investigation. Application of new MR brain neuroimaging sequences sensitive to iron (SWI, Susceptibility Weighted Imaging) represents a significant improvement over conventional gradient echo T₂* for early recognition and diagnosis of CAA and MH. (FIG. 1)

During the past three years the cognitive course of 76 mildly cognitively impaired (MCI) and 28 control participants has been correlated to both SWI MH detection and serum proteomic tests developed by Dr. Lance Liotta. Sixteen MCI cases have progressed to dementia (Alzheimer's disease 15% per annum conversion) and 6 of the 16 show a progressive increase of MH (>10) in patterns consistent with cerebral amyloid angiopathy (CAA). All 6 MH cases have unique low molecular weight (LMW) serum proteomic biomarkers. SWI imaging for MH detection will be enhanced by 3T scanners being installed in 2007. Detection limits of variably sized brain MH are given in Table 1.

TABLE 1
Detection of Variably Sized Brain MH

| MH Size O.D. | | Detection Method |
|---|---|---|
| 50-200 | μm | Light microscope histology |
| ~50 to 500 | μm | ? SWI at 3T |
| ~1-10 | mm | SWI at 1.5T |
| ~3-10 | mm | GE-T₂* 1.5T |
| >1 | cm | Conventional CT, T₁, T₂, MR |

Cognitive loss is secondary to neuronal and neuropil damage in a larger MH perifocal reactive zone secondary to overexpressed brain heme-oxygenase-1 (HO-1). A progressive increase of brain MH associated with CAA is a significant cause for sporadic late onset cognitive loss and can be diagnosed earlier and more precisely with SWI MR imaging.

High field MR should provide an earlier and more sensitive detection of MH (CAA). MH counts will be made by blinded, experienced neuroradiologists and readers at LLUMC and DMRI. Sequential proteomic studies of participant serum will be conducted at GMU by Dr. Liotta's group. Dr. Vinters' Neuropathology resource (UCLA) will provide both frozen and formalin fixed CAA brains for study by both SWI imaging (LLUMC) and laser capture microdissection (LCM) at GMU. LCM will enable determination of gradients of neuronal and neuropil destruction, heme distribution, heme oxygenase activation, apoptosis, and other critical substrates.

### Example 4

### Selection of HO-1 Targeting siRNAs

The sequences of HO-1 targeting siRNAs were checked for theoretical specificity against the mouse transcriptome by blast searches against the mouse genome using NCBI. Five different siRNA sequences were accepted, as well as one nonspecific siRNA scrambled duplex. The following steps will be taken to maximize success in siRNA target sequence selection. (1) Find the regions of a cDNA to choose target sequences. A target sequence must be specific to the target gene and show no significant homology to any other genes. Using the blast search, regions of the target cDNA with no or low homology to other genes can be identified, from which candidate siRNA target sequences can be chosen. (2) A target sequence should start with a “G.” RNA Polymerase III always starts its transcription with a “G” from the U6 promoter. Therefore, one needs to find a region that begins with a “G” as a target sequence candidate. (3) Do not leave any string of four “Ts” in the designed hairpin. Four or five “Ts” is a stop signal for the transcription of Pol III and their presence in the designed hairpin will lead to premature transcriptional termination. (4) Avoid sequences containing KpnI or HindIII sites. KpnI and HindIII are used to digest the PCR products later on. Their presence in a target sequence will result in nonfunctional constructs. (5) Avoid sequences close to the ATG translational start codon. The region close to ATG on the mRNA may be associated with multiple proteins involved in translation that may interfere with RISC binding. A target sequence can also be selected from a 3″-UTR region. (6) Avoid sequences with internal repeats or palindromes. The presence of these structures will reduce the production of functional hairpins. (7) Use a sequence with a low G/C content, especially at its 3′ end. SiRNAs with lower G/C content are believed to yield better silencing. (8) Use a sequence with high specificity to the target gene. All target sequence candidates need to be analyzed using the NCBI/Blast website to ensure that they do not significantly match any other gene sequence.

siRNAs will be designed and tested for maximal knockdown efficacy. Our sequences of choice at present are described below. Testing as described above will commence upon grant funding.

The design of siRNAs is based on the characterization of siRNA by Elbashir S M et al. Harborth J. et al., Antisense Nucleic Acid Drug Dev. April 2003; 13(2):83-105; Harborth J. et al., J Cell Sci. December 2001; 114(Pt 24):4557-4565. SiRNAs with stability modifications for in vivo use (siSTABLE) will be synthesized in the 2′-deprotected, duplexed, desalted, and purified form by Dharmacon Research, Inc. (Lafayette, Colo.). The sense and antisense strands of mouse HO-1 siRNA are: sequence 1, 5′-AAGGACAUGGCCUUCUGGUAUdTdT-3′ (sense) (SEQ ID NO: 1) and 5′-AUACCAGAAGGCCAUGUCCUUdTdT-3′ (antisense) (SEQ ID NO: 2); sequence 2, 5′-AAUGAACACUCUGGAGAUGACdTdT-3′ (sense) (SEQ ID NO: 3) and 5′-GUCAUCUCCAGAGUGUUCAUUdTdT-3′ (antisense) (SEQ ID NO: 4); sequence 3, 5′-AAGACCAGAGUCCCUCACAGAdTdT-3′ (sense) (SEQ ID NO: 5) and 5′-UCUGUGAGGGACUCUGGUCUUdTdT-3′ (antisense) (SEQ ID NO: 6); sequence 4, 5′-AAGCCACACAGCACUAUGUAAdTdT-3′ (sense) (SEQ ID NO: 7) and 5′-UUACAUAGUGCUGUGUGGCUUdTdT-3′ (antisense) (SEQ ID NO: 8); sequence 5, 5′-AAGCCGAGAAUGCUGAGUUCAdTdT-3′ (sense) (SEQ ID NO: 9) and 5′-UGAACUCAGCAUUCUCGGCUUdTdT-3′ (antisense) (SEQ ID NO: 10). Nonspecific siRNA scrambled duplex (sense, 5′-GCGCGCUUUGUAGGAUUCGdTdT-3′ (SEQ ID NO: 11); antisense, 5′-CGAAUCCUACAAAGCGCGCdTdT-3′) (SEQ ID NO: 12) will also be synthesized by Dhammacon Research, Inc. SiRNAs will all be screened for their in vitro knockdown efficiency prior to in vivo use using RT-PCR and Western blotting techniques in a HO-1 expressing cell culture system. Suttner D. M., et al., Faseb J October 1999; 13(13):1800-1809.

### Example 5

### In vitro Testing of HO-1 siRNA

This Example illustrates screening of siRNAs for their in vitro knockdown efficiency prior to in vivo use using RT-PCR and Western blotting techniques in a HO-1 expressing cell culture system as described below.

SiRNAs that reveal the highest efficiency (a consistently maximal knockdown of greater than ˜90%) will be chosen for the in vivo experiments. In vitro testing of the selected siRNAs will be done using a recently developed DNA vector-based technology that produces functional double-stranded siRNAs to suppress gene expression in mammalian cells as previously described.⁽⁴²⁾ Briefly, the pBS/U6 expression vector⁽⁴³⁾ will be used for all subsequent subcloning experiments. A pair of 21-23 nucleotides of DNA (containing the target sequence) with a palindrome symmetric structure linked by a short loop (6-9 nucleotides) will be inserted downstream of the U6 promoter. These siRNA plasmids will be introduced into cells using Lipofectamine 2000 (Invitrogen) transfection approaches.

Two to three days after transfection, gene silencing will be monitored using immunofluorescence, Western blotting and PCR. Cells will be co-transfected by the siRNA plasmid and a second plasmid encoding green fluorescence protein (GFP) and a third plasmid encoding an HA-epitope tagged HO-1. The cells on the cover slip will be stained with antibody recognizing the target protein HO-1, followed by blotting with fluorescence dye conjugated secondary antibody. If the siRNA plasmid is effective, the signal for the target gene will significantly decrease in the GFP positive cells. If high transfection efficiency can be achieved, the silencing of the targeted endogenous gene can be visualized by Western blotting using the anti-HO-1 antibody or by identification of the transcript using RT-PCR. If the transfection efficiency is low, Western blot may not be able to detect the expression difference of the endogenous target gene. However, the efficacy of siRNA construct can be determined by Western blot on the suppression of the expression of the co-transfected target gene tagged by an epitope.

### Example 6

### In vivo Testing of siRNA

This Example illustrates in vivo testing of siRNA for tolerance.

After siRNAs are tested for their gene knockdown ability in vitro, sequences will be submitted for chemical synthesizing from, for example, Dharmacon Research, Inc. These chemically synthesized siRNAs will then be used in vivo. Osmotic minipumps (Alzet model 1004, Cupertino, CA) will be filled to infuse HO-1-siRNA or scrambled-siRNAs for 4 weeks. This time frame was chosen on the basis of previous studies showing that a maximally effective RNAi response requires 2 weeks of siRNA infusion. Thakker D. R. et al, Proc Natl Acad Sci USA. Dec. 7 2004; 101(49):17270-17275. Signs of tolerance will be carefully monitored.

### Example 7

### Stereotactic Procedure for Intraventricular Infusion, Osmotic Pumps

The stereotactic surgical procedure for implantation of the cannula into the dorsal third ventricle, cannulation and subcutaneous placement of the Alzet pump is established. Hoyer D. et al., J Receptors and Signal Transduction. 2006; 26:527-547.

The animal will be anesthetized for placement of the cannula. Day 1 of the start of the infusion will be designated as day 0. The cannula is placed into the dorsal third ventricle with the following stereotactic coordinates: AP −0.5 mm; ML: 0 mm, DV: −3 mm, relative to the bregma according to the stereotactic atlas of Paxinos and Franklin. Paxinos and Franklin, The Mouse Brain in Stereotaxic Coordinates. 2nd ed. San Diego: Academic Press; 2001.

Osmotic minipumps (Alzet model 1004, Durect Corporation, Cupertino, Calif., USA) will be filled as per the manufacturers instruction in order to infuse vehicle (2.64 μl/day), HO-1-siRNA or nonspecific siRNA (0.4 mg/day) for 4 weeks. This duration of infusion was chosen based on previous studies by Thakker D. R. et al. (Cryan et al., Biochem Soc Trans. April 2007; 35(Pt 2):411-415. Thakker D. R. et al., Pharmacol Ther. March 2006; 109(3):413-438.) using the Alzet model 1002 osmotic minipumps, showing that a maximally effective RNAi response in mice requires 2 weeks of siRNA infusion. This original work was limited by the minipump model 1002, as it was only capable of a 2 week period of infusion. With the new model 1004, siRNAs will be deliverable up to 4 weeks. Using a lower dose of siRNA over a longer period of time will allow for greater knockdown and lower toxicity. A maximally effective dose of siRNA will be used that is well tolerated with no signs of neurotoxicity (hind-limb paralysis, vocalization, food intake or neuroanatomical damage) following i.c.v. application for 4 weeks.

### Example 8

### Primary and Secondary Outcomes

The Barnes Maze tests spatial learning and memory after the mouse learns the special location of the target box. “Outcome” is the amount of time required for the animal to locate the safe box, with results analyzed by repeated measures of analysis of variance (ANOVA). This spatial memory test assesses ability to learn and remember the location of an escape box over the course of a 5-day period and is a widely accepted technique to assess cognitive status in mice. Performance of each animal for each testing is the average latency of two trials. The Barnes Maze reliably detects spatial memory deficits in the Tg-SwDI transgenic animals as early as 3 months of age compared with wild type controls, with the deficits increasing at 12 months. Fan R. et al., J Neurosci. Mar. 21 2007; 27(12):3057-3063.

Results of Barnes Maze testing are expressed in seconds (latency) to find the escape box (M±S.E.M., wild type=20±10), (Tg-SwDI=95±15 sec). See references in appendix for statistical and outcome interpretation. This is the primary outcome of the experiment. Proof of gene knockdown are secondary outcomes.

Hemispheres fixed in ethanol polyethylene glycol will be screened by immunostaining to quantitate both inflammatory response and numbers of HO-1 immunopositive reactive astrocytes and microglia. Staining protocols are known in the art and also described below. Tissue sections will be reviewed for a blinded analysis of the amyloid burden, iron deposition, inflammatory process quantitation, and neuronal damage. Immunohistochemistry procedures are known in the art and described in, for example, Xu, F. et al., Neuroscience. Apr. 27, 2007; 146(1):98-107. Snap frozen tissue will be studied separately.

### Example 9

### Quantitation of HO-1 Participation in the Inflammatory Response

### Quantitative Analysis of Reactive Astrocytes and Activated Microglia Densities

Total numbers of reactive astrocytes, activated microglia, HO-1 immunopositive cells in the fronto-temporal cortex, CA1 and CA2 fields of the hippocampus, thalamus, and subiculum regions will be estimated using the Stereologer software system (Systems Planning and Analysis) as described. Long J. M. et al., J Neurosci Methods. Oct. 1, 1998; 84(1-2):101-108; Miao J. et al., J Neurosci. Jul. 6, 2005; 25(27):6271-6277; Miao J. et al., Am J Pathol. August 2005; 167(2):505-515. Briefly, every 10th section is selected and generated 10 to 15 sections per reference space in a systematic-random manner. Immunopositive cells are counted using the optical fractionator method with the dissector principle and unbiased counting rules. Criteria for counting cells requires that cells exhibited positive immunostaining (HO-1, GFAP for astrocytes and mAb to I-A/I-E MHC class II alloantigens or mAb 5D4 to keratan sulfate for activated microglia) and morphological features consistent with each cell type.

### Immunohistochemical Analysis

Immunostainings will be performed on de-paraffined sections or free-floating sections. Antigen retrieval is performed by treatment with proteinase K (0.2 mg/ml) for 5 min at room temperature for Aβ, and collagen type IV immunostaining, or in 1:100 antigen-unmasking solution (Vector Lab) for 30 min at 90° C. in a water-bath for activated microglia immunostaining with 5D4 antibody or in 10 mM sodium citrate, pH 6.0 for 30 min at 90° C. for MHCII microglial staining. Nonspecific binding is blocked by incubating in PBS containing 0.1% Triton X-100 and 2% bovine serum albumin (Sigma-Aldrich) for 20 min at room temperature. Primary antibodies are incubated with the brain sections overnight at 4° C. and detected with horseradish peroxidase-conjugated or alkaline phosphatase-conjugated secondary antibodies. Alternatively, peroxidase-conjugated streptavidin in conjunction with biotinylated secondary antibody will be used for detecting microglia. Peroxidase activity is visualized either with a stable diaminobenzidine solution (Invitrogen, Carlsbad, Calif.) or with the fast red substrate system (Spring Bioscience, Fremont, Calif.), respectively, as substrate. Thioflavin-S staining for fibrillar amyloid is performed as described. Dickson D. W. et al., Acta Neuropathol (Berl). 1990; 79(5):486-493. The following antibodies will be used for immunostaining: monoclonal antibody 66.1 (1:250), which recognizes residues 1 to 5 of human Aβ (Deane R. et al., Nat Med. July 2003; 9(7):907-913), rabbit polyclonal antibody to collagen type IV (1:100; Research Diagnostics Inc., Flanders, N.J.); monoclonal antibody to glial fibrillary acidic protein (GFAP) for identification of astrocytes (1:1000, Chemicon); monoclonal antibody 5D4 to keratan sulfate for identification of activated microglia (1:300; Seikagaku Corporation, Japan) and monoclonal antibody to MHC class II (1:200; BD Pharmingen, San Jose, Calif.) for identification of activated microglia; monoclonal antibody to HO-1 (1:100) Biomol and biotinylated goat anti-mouse IgG (1:200) and ABC kit (Vector Laboratories, Burlingame, Calif.) according to the manufacturer's recommendations.

### Protocols for HO-1 and HO-2 Immunohistochemistry Staining

The primary antibody incubation is with a rabbit polyclonal antibody to HO-1 or to HO-2 (Biomol 1:100), then incubated with anti-rabbit IgG—Biotin antibody (Chemicon 1:1000) incubated with ABC Reagent (Vector) and Stable DAB. The protocol for HO-1 and HO-2—GFAP was first blocking with superblock blocking buffer, primary antibody incubation with rabbit polyclonal antibody to HO-1 or HO-2 (Biomol 1:100) plus mouse anti-GFAP (Chemicon, 1:1000) followed with incubation with Alexa Fluor donkey anti-rabbit IgG (Molecular Probes, 1:1500)+Alexa Fluor 596 donkey anti-mouse IgG (Molecular Probes, 1:1500).

The cell counts are expressed as n×10³ cells/mm³, baseline Tg-SwDI counts (1 year old mice) for n=10 cortex, 70 in hippocampus, thalamus, subiculum, WT from fourfold to tenfold less. The HO-1 GFAP immunopositive reactive astocyte/microglia in the untreated Tg-SwDI animals are anticipated to be 40×10³/mm³, none anticipated in the WT. As noted the regional differences noted on tissue staining of HO-1 immunopositive cells will dictate the number of LCM cases to be studied.

### Example 10

### Laser Capture Microdissection

Laser Capture Microdissection (LCM) will be conducted on formalin fixed paraffin embedded and ethanol fixed tissue. The complete protocol for conducting LCM is provided in Espina et al., Nat Protoc. 2006; 1(2):586-603. The procured cells will be lysed and analyzed by Reverse Phase Protein microarrays (RPAs) following published protocols.⁽⁵⁵⁾ The analytic precision is less than 7.5 percent. HO-1 will be the primary analyte to be measured. Microdissection will be conducted at a series of radial distances surrounding vessels with amyloid angiopathy, in regions of peri-adventitial inflammatory microglial cells and astrocytes.

### Biochemical Studies on the Snap-Frozen Brain Powder

Preparing a frozen brain powder of snap-frozen hemispheres will enable global evaluation of HO-1 gene knockdown. Tissue will be powdered with mortar and pestle under liquid nitrogen, three 4-5 mg powder aliquots will be obtained from each hemisphere, and different extraction procedures used depending on the desired outcome measure.

### HO-1 Gene Knockdown: RT-PCR, Western Blots

After excision, brain tissue for RNA and protein extraction will be frozen in liquid nitrogen until needed. When needed, liquid nitrogen will be added to the tissue in a mortar after which the tissue will be powdered using a mortar and pestle. For RNA extraction, powder will be next homogenized in TRI REAGENT as per the manufacturers protocol (Molecular Research Center, Inc., Cincinnati, Ohio) at a volume of 1 ml/50 mg tissue. For protein extraction, RIPA buffer (1% Igepal CA-630 (0.5 ml), 0.5% Sodium deoxycholate (0.25 g), 0.1% SDS (0.05 g), PBS (49.5 ml)) will be added to the powdered tissue, vortexed for 60 seconds, put on ice for 45 minutes, and again homogenized with a polytron homogenizer (2×15 seconds). Material will be centrifuged at 12,000 g for 10 minutes at 4° C. and the supernatant will be kept for further identification. Approximately 5 ml RIPA per gram of tissue will be used.

### Quantitative Real-Time RT-PCR Analysis

Total RNA will be extracted from cells and followed by reverse-transcription with a first-strand RT-PCR kit (Invitrogen) per manufacture's instructions. PCR will be performed with the LightCycler® RNA Master SYBR Green I using the LightCycler® 2.0 System (Roche). To detect the induction of HO-1 and HO-2 the following primers will be used: for HO-1 (forward primer: 5′-caggacatggccttctggta-3′; reverse primer: 5′-tgtcgatgttcgggaaggta-3′); for HO-2 (forward primer: 5′-caaggaccacccagccttcg-3′; reverse primer: 5′-cccagtgctgggaagttttg-3′) and primers to b-actin will be used as control (forward primer: 5′-ccggcatgtgcaaagccggc-3′; reverse primer: 5′-tggggtgttgaaggtctcaa-3′). The cycle quantity required to reach a threshold in the linear range (Qt) will be determined and compared with a standard curve for each primer set generated by five 3-fold dilutions of the first-strand cDNA of known concentration. Data will be represented as the mean±S.D. of normalized activities of HO-1 and HO-2 relative to that of β-actin in each treatment.

Western blotting will be utilized to determine extent of HO-1 gene knockdown. The brain homogenates will be separated into cytosolic and particulate fractions, cytosolic fractions loaded onto 10% Bis-Tris gel and transferred to Millipore membranes and probed with the HO-1 and HO-2 mabs. Blots will be visualized by enhanced using fluorescently-labeled secondary antibodies and analyzed on the Odyssey System. The Western blot analysis will be used to document extent of HO-1 gene silencing as well as HO-2 activity.

### Example 11

### Levels of Heme-Oxygenase 1 are Elevated in Vitreous Humor of “Wet” Macular Degeneration

This Example illustrates use of Reverse Phase Protein Microarrays to detect elevated levels of heme-oxygenase 1 in the vitreous humor of “wet” macular degeneration cases. A Heme-Oxygenase-1 (HO-1) antibody was used on vitreous samples printed on our Reverse Phase Protein Microarrays.

Twenty-six vitreous samples were collected from patients after informed consent was obtained following an IRB approved protocol and adhering to the tenets of the Declaration of Helsinki. Control samples were collected from surgical patients immediately prior to pars plana vitrectomy (n=7) for the following indications: macular hole, epiretinal membrane, or retinal detachment. Nineteen samples were collected from patients with wet age-related macular degeneration, idiopathic choroidal neovascularization or diabetic retinopathy. Patients underwent vitreous sampling in the office prior to intravitreal injection.

In each case, a topical anesthetic followed by additional anesthetic was applied to the pars plana via a cotton pled-get. A sterile eyelid speculum exposed the pars plana. Betadine 5% was applied to the pars plana and formix to achieve sterility. A 1 cc syringe with a 25 gauge needle was used to obtain a small quantity (0.05 to 0.2 cc) of liquid vitreous, being careful to avoid aspiration of any subconjunctival or surface fluid while withdrawing the needle from the eye. All specimens were frozen at −20DC for storage until subsequent analysis by reverse phase protein microarrays.

Patients were characterized by disease process, as active neo-vascularization (n=19), or non neOvascularization (n=7).

### Reverse Phase Protein Microarrays (RPPM)

Protein Microarray Construction: Total protein content of the vitreous samples was measured spectrophotometricly (Bradford method). The samples were diluted in extraction buffer (T-PER (Pierce, Indianapolis, Ind.), 2-mercaptoehtanol (Sigma, St. Louis, Mo.) and 2×SDS Tris-glycine loading buffer (Invitrogen, Carlsbad, Calif.)) and denatured by heating for 8 minutes at 100DC prior to dilution in the microtiter plate. Briefly, the lysates were printed on glass backed nitrocellulose array slides (FAST Slides Whatman, Florham Park, N.J.) using an Aushon 2470 arrayer (Aushon BioSystems, Burlington, Mass.) equipped with 350 !lm pins. Each lysate was printed in a dilution curve representing neat, 1:2, 1:4, 1:8, 1:16 dilutions. The slides were stored with desiccant (Drierite, W. A. Hammond, Xenia, Ohio) at −20DC prior to immunostaining.

Control Microarrays: Cellular lysates prepared from A431::I:: EGF, HeLa::1:: Pervanadate, Human Endothelial ::I:: Pervanadate (Becton Dickinson, Franklin Lakes, N.J.) and CHO-T ::I:: Insulin (Biosource/Invitrogen, Carls bad, CA) were printed on each array for quality control assessments. Human Endothelial::1:: Pervanadate cellular lysates were printed on arrays for sensitivity and precision comparisons.

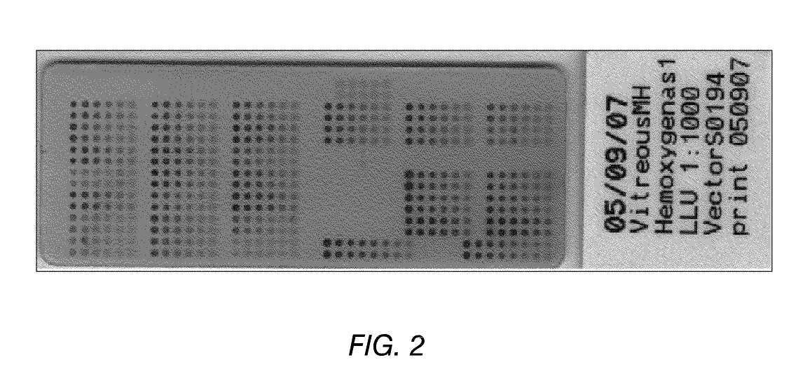

Protein Microarray Immunostaining: Immunostaining was performed on an automated slide stainer per manufacturer's instructions (Auto stainer CSA kit, Dako, Carpinteria, CA). The slide was incubated with a single primary antibody at room temperature for 30 minutes (HemeOxygenase-1 (C. Mueller, Loma Linda University)). A negative control slide was incubated with antibody diluent. Secondary antibody was goat anti-rabbit IgG H+L (1:5000) (Vector Labs, Burlingame, Calif.). Total protein per microarray spot was determined with a Sypro Ruby protein stain (Invitrogen/Molecular Probes, Eugene, Oreg.) per manufacturer's directions and imaged with a CCD camera (Alpha Innotech, San Leandro, Calif.). The RPPM immunostained with anti-Heme-Oxygenase-1 is shown in FIG. 2.

Bioinformaties method for micro array analysis: Each array was scanned, spot intensity analyzed, data normalized, and a standardized, single data value was generated for each sample on the array (Image Quant v5.2, GE Healthcare, Piscataway, N.J.). Spot intensity was integrated over a fixed area. Local area background intensity was calculated for each spot with the unprinted adjacent slide background. This resulted in a single data point for each sample, for comparison to every other spot on the array. Each sample was printed in duplicate in a miniature dilution curve. All the data was analyzed to derive a concentration value averaged between the replicates and within the linear range of the dilution curve.

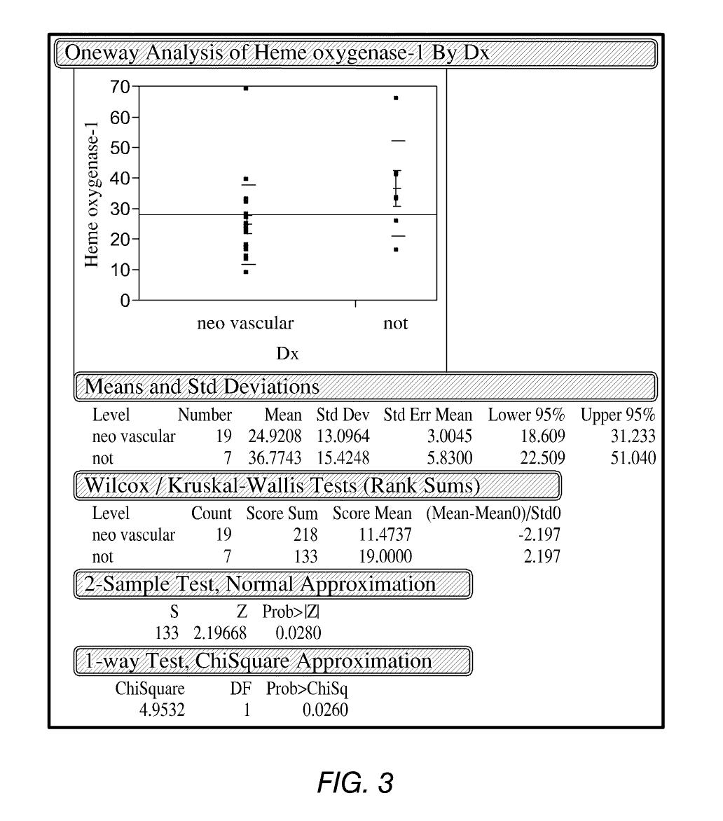

Statistics: Wilcoxon Rank Sum analysis was used to compare values between two groups. (FIG. 3) P values less than 0.05 were considered significant. Non-parametric correlations were compared using Spearman's Rho analysis (see, Table 2 below).

### Results

Heme Oxygenase-1 was significantly associated with caspase 8, MMP-9 and PDGFRb Y716 in the neovascular disease process group. Table 2 below depicts Spearman's non-parametric correlation of Heme-Oxygenase-1 with other selected proteins analyzed in the vitreos samples. The samples were categorized by disease process, as neo-vascular disease (we AMD, choroidal neovascularization, or diabetic retinopathy) versus non-neovascular disease (macular hole, epi-retinal membrane or retinal detachment).

TABLE 2
Spearman's Rho non-parametric correlation of HO-1 with other selected
proteins analyzed in vitreous samples

| Neovascular Disease | | | | Non-neovascular Disease | | | |
|---|---|---|---|---|---|---|---|
| | | Spearman | | | | Spearman | |
| Variable | By Variable | Rho | Prob > \|Rho\| | Variable | By Variable | Rho | Prob >\|Rho\| |
| Heme | Caspase 8 | 0.828070175 | 1.2015E−05 | Heme | Caspase 8 | 0.428571429 | 0.337368311 |
| oxygenase-1 | | | | oxygenase-1 | | | |
| MMP-9 | Heme | 0.971929825 | 4.05535E−12 | MMP-9 | Heme | 0.892857143 | 0.006807187 |
| | oxygenase-1 | | | | oxygenase-1 | | |
| mTOR | Heme | 0.80877193 | 2.76722E−05 | mTOR | Heme | 0.214285714 | 0.644511581 |
| ser2481 | oxygenase-1 | | | ser2481 | oxygenase-1 | | |
| PDGFRB | Caspase 8 | 0.522807018 | 0.021636911 | PDGFRB | Caspase 8 | 0.892857143 | 0.006807187 |
| Y716 | | | | Y716 | | | |
| PDGFRB | Heme | 0.821052632 | 1.64583E−05 | PDGFRB | Heme | 0.642857143 | 0.119392373 |
| Y716 | oxygenase-1 | | | Y716 | oxygenase-1 | | |

### Example 12

### Therapeutic Trial of HO-1, HO-2, MMP, Caspase Inhibitor and/or Metalloporphyrin Inhibitors in the Mouse Model of Macular Degeneration

A mouse model of macular degeneration will be studied for the therapeutic effects of agents directed to brain HO-1, HO-2, MMP, caspase inhibitor and/or metalloporphyrin inhibition.

A mouse model for macular degeneration will be evaluated using neurologic, pathologic, and biochemical parameters. A number of mouse models for macular degeneration are available, and include, for example, the ELOVL4 transgenic mouse (Karan et al., PNAS, 2005; Vol. 102, No. 11:4164-4169), the Bst mouse, Cc1-2 mouse, or the Abca4 knockout mouse.

SiRNAs tested for their gene knockdown ability in vitro, as described above in Example 5 and 6 will be used in vivo. The siRNA can be chemically synthesized, or prepared in any of the ways described above. In some embodiments, the siRNA can be expressed from an expression construct. For introduction to the eye, intravitreous or periocular injection can be used to administer HO-1-siRNA or scrambled-siRNAs. See, for example, Campochiaro, Gene Therapy, 2006, 13 559-562. In other embodiments, an implantable delivery device may be used to infuse HO-1-siRNA or scrambled-siRNAs. The treatment period may vary and in some embodiments, can be about 4 weeks. Signs of tolerance will be carefully monitored. Day 1 of the start of the injection or infusion will be designated as day 0.

A maximally effective dose of siRNA will be used that is well tolerated with no signs of neurotoxicity (hind-limb paralysis, vocalization, food intake or neuroanatomical damage) following application for 4 weeks.

Retinas will be screened by immunostaining to quantitate both inflammatory response and numbers of HO-1 immunopositive reactive cells. Tissue sections will be reviewed independently for a blinded analysis of the amyloid burden, iron deposition, inflammatory process quantitation, and cellular damage.

Western blotting will be utilized to determine extent of HO-1 gene knockdown. The Western blot analysis will be used to document extent of HO-1 gene silencing as well as HO-2 activity.

In addition, Reverse Phase Protein Microarrays will be used to detect levels of HO-1 and HO-2 in the vitreous humor of treated and control animals. HO-1 and HO-2 antibodies will be used on vitreous samples printed on our Reverse Phase Protein Microarrays. Analysis will be conducted, for example, as described above in Example 11.

### CONCLUSION

All patents and publications are herein incorporated by reference in their entireties to the same extent as if each individual publication was specifically and individually indicated to be incorporated by reference.

The invention illustratively described herein suitably can be practiced in the absence of any element or elements, limitation or limitations that is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

1. A method of treating or inhibiting progress of dementia in a mammal, comprising administering an inhibitor of heme oxygenase-1 (HO-1) or heme oxygenase-2 (HO-2) in a manner that permits access to brain sites of said mammal.

2. The method according to claim 1, wherein said inhibitor is an siRNA.

3. The method according to claim 2, wherein said siRNA is in a liposome.

4. The method according to claim 3, wherein said liposome is targeted to an endothelial cell receptor.

5. The method according to claim 4, wherein said endothelial cell receptor is an LDL receptor.

6. The method according to claim 1, wherein the administration is intravenous.

7. The method according to claim 6, wherein the administration is carried out using an osmotic pump.

8. The method according to claim 2, wherein said liposome is a 1,2-dioleoyl-sn-glycero-3-phosphatidylcholine (DOPC) liposome.

9. The method according to claim 1, wherein the inhibitor is administered to cerebrospinal fluid (CSF) of the mammal.

10. The method according to claim 9, wherein the administration is via lumbar puncture.

11. The method according to claim 9, wherein the administration is via ventricular puncture.

12. The method according to claim 8, wherein said liposome is administered intravenously using an osmotic pump.

13. The method according to claim 12, wherein said DOPC liposome is targeted to an LDL receptor.

14. The method according to claim 1, wherein said mammal is an elderly individual having fragile microvessels.

15. The method according to claim 1, wherein said mammal has Alzheimer's disease.

16. A method of treating or inhibiting progress of dementia in a mammal, comprising administering an inhibitor of HO-1 or HO-2 to the brain of said mammal.

17. The method according to claim 16, wherein said inhibitor is an siRNA.

18. The method according to claim 17, wherein said siRNA is in a liposome.

19. The method according to claim 18, wherein said liposome is targeted to an endothelial cell receptor.

20. The method according to claim 19, wherein said endothelial cell receptor is an LDL receptor.

21. The method according to claim 16, wherein the administration is intravenous.

22. The method according to claim 21, wherein the administration is carried out using an osmotic pump.

23. The method according to claim 22, wherein said osmotic pump is an ALZET® osmotic pump.

24. The method according to claim 17, wherein said siRNA is in a 1,2-dioleoyl-sn-glycero-3-phosphatidylcholine (DOPC) liposome that is administered intravenously using an ALZET® osmotic pump.

25. The method according to claim 24, wherein said DOPC liposome is targeted to an LDL receptor.

26. The method according to claim 16, wherein said mammal is an elderly individual having fragile microvessels.

27. The method according to claim 16, wherein said mammal has Alzheimer's disease.

28. (canceled)

29. The method according to claim 1, wherein said inhibitor is a metalloporphyrin.

30-34. (canceled)

35. The method according to claim 29, wherein said metalloporphyrin is administered to a blood vessel endothelial cell receptor of said mammal, thereby inhibiting HO-1 and HO-2 and preventing weakening and bleeding in the vessel wall.

36. A method of treating or inhibiting progress of macular degeneration in a mammal, comprising administering a composition comprising an inhibitor of heme oxygenase-1 (HO-1) or heme oxygenase-2 (HO-2) in a manner that permits access to a macula of said mammal.

37. The method according to claim 36, wherein the administering is by intravitreal injection.

38. The method of claim 36, wherein said inhibitor is an siRNA.

39. The method of claim 38, wherein said siRNA is in a liposome.

40. The method according to claim 39, wherein said liposome is targeted to an endothelial cell receptor.

41. The method according to claim 40, wherein said endothelial cell receptor is an LDL receptor.

42. The method according to claim 36, wherein said inhibitor is a metalloporphyrin.


SEQUENCE LISTING (12 sequences)

SEQ ID NO: 1 - RNA, 23 nt, Artificial Sequence
  HO-1 siRNA sequence
aaggacauggccuucugguaunn

SEQ ID NO: 2 - RNA, 23 nt, Artificial Sequence
  HO-1 siRNA sequence
auaccagaaggccauguccuunn

SEQ ID NO: 3 - RNA, 23 nt, Artificial Sequence
  HO-1 siRNA sequence
aaugaacacucuggagaugacnn

SEQ ID NO: 4 - RNA, 23 nt, Artificial Sequence
  HO-1 siRNA sequence
gucaucuccagaguguucauunn

SEQ ID NO: 5 - RNA, 23 nt, Artificial Sequence
  HO-1 siRNA sequence
aagaccagagucccucacagann

SEQ ID NO: 6 - RNA, 23 nt, Artificial Sequence
  HO-1 siRNA sequence
ucugugagggacucuggucuunn

SEQ ID NO: 7 - RNA, 23 nt, Artificial Sequence
  HO-1 siRNA sequence
aagccacacagcacuauguaann

SEQ ID NO: 8 - RNA, 23 nt, Artificial Sequence
  HO-1 siRNA sequence
uuacauagugcuguguggcuunn

SEQ ID NO: 9 - RNA, 23 nt, Artificial Sequence
  HO-1 siRNA sequence
aagccgagaaugcugaguucann

SEQ ID NO: 10 - RNA, 23 nt, Artificial Sequence
  HO-1 siRNA sequence
ugaacucagcauucucggcuunn

SEQ ID NO: 11 - RNA, 21 nt, Artificial Sequence
  HO-1 siRNA sequence
gcgcgcuuuguaggauucgnn

SEQ ID NO: 12 - RNA, 21 nt, Artificial Sequence
  HO-1 siRNA sequence
cgaauccuacaaagcgcgcnn